SEQUENCE LISTING (16 sequences)

SEQ ID NO: 1 - DNA, 18 bp, ARTIFICIAL SEQUENCE
  SYNTHESIZED
ccctgaccccctacctga

SEQ ID NO: 2 - DNA, 20 bp, ARTIFICIAL SEQUENCE
  SYNTHESIZED
cttgggcatagccagcatgt

SEQ ID NO: 3 - DNA, 20 bp, ARTIFICIAL SEQUENCE
  SYNTHESIZED
caggaggcttgtgaggccta

SEQ ID NO: 4 - DNA, 20 bp, ARTIFICIAL SEQUENCE
  SYNTHESIZED
agctggatgtctttgggcat

SEQ ID NO: 5 - DNA, 20 bp, ARTIFICIAL SEQUENCE
  SYNTHESIZED
ggttgcagaagtgccgaaag

SEQ ID NO: 6 - DNA, 20 bp, ARTIFICIAL SEQUENCE
  SYNTHESIZED
gtaacttgccaatccacggg

SEQ ID NO: 7 - DNA, 20 bp, ARTIFICIAL SEQUENCE
  SYNTHESIZED
tgatcccatcactgtgacgg

SEQ ID NO: 8 - DNA, 20 bp, ARTIFICIAL SEQUENCE
  SYNTHESIZED
gctttttgcggtattcctgc

SEQ ID NO: 9 - DNA, 20 bp, ARTIFICIAL SEQUENCE
  SYNTHESIZED
agaccttcgtgcaggcaatc

SEQ ID NO: 10 - DNA, 20 bp, ARTIFICIAL SEQUENCE
  SYNTHESIZED
aggaaagccaccgccaatat

SEQ ID NO: 11 - DNA, 20 bp, ARTIFICIAL SEQUENCE
  SYNTHESIZED
catcactccagaaccagaat

SEQ ID NO: 12 - DNA, 20 bp, ARTIFICIAL SEQUENCE
  SYNTHESIZED
taacaaacaaggtggtcaca

SEQ ID NO: 13 - DNA, 20 bp, ARTIFICIAL SEQUENCE
  SYNTHESIZED
cctccactgctgtagtaacc

SEQ ID NO: 14 - DNA, 20 bp, ARTIFICIAL SEQUENCE
  SYNTHESIZED
gaaagattgtgatccctctg

SEQ ID NO: 15 - DNA, 20 bp, ARTIFICIAL SEQUENCE
  SYNTHESIZED
aacctgccaaatatgatgac

SEQ ID NO: 16 - DNA, 20 bp, ARTIFICIAL SEQUENCE
  SYNTHESIZED
ttgaagtcagaggagaccac

(19) United States
(12) Patent Application Publication
(10) Pub. No.: US 2008/0009001 A1
(43) Pub. Date: Jan. 10, 2008

(54) Method for Identification of Neoplastic Transformation with Particular Reference to Prostate Cancer

(76) Inventors: Saverio Bettuzzi, Montecchio Emilia (IT); Arnaldo Corti, Pontecchio (IT)

Correspondence Address: JACOBSON HOLMAN PLLC, 400 SEVENTH STREET N.W., SUITE 600, WASHINGTON, DC, 20004, US

(21) Appl. No.: 11/664,792

(22) PCT Filed: Oct. 4, 2005
(86) PCT No.: PCT/IB05/02942

§ 371 Date: Apr. 6, 2007

(30) Foreign Application Priority Data
Oct. 6, 2004 (IT) BZ 2004 A 000 048

(51) Int. Cl. C12Q 1/68

(52) U.S. Cl. 435/6

(57) ABSTRACT

A method used to design a microchip (DNA microarray) for identifying the presence of prostate tumor, evaluating its degree of mali-gnity (typization, characterization) and supplying information allowing prediction of the clinical course of the illness (prognosis) is discussed below. The method is based on assessment of the levels of expression of a definite package of genes in the tumoral tissue in comparison with the corresponding benign tissue. The readings thus obtained, - alone, in different combinations with each other or in different combinations and integrated with standardized clinical data - give the results described above.

This invention relates to a method for identification of neoplastic transformation with particular reference to prostate cancer in accordance with the classifying part of claim 1. In particular the method concerns identification of a group of genes whose expression levels, however determined, even after integration with other data of clinical origin, is informative for evaluation of the transformation of the tumoral transformation of the prostate tissue, of its degree of malignancy and for the prognosis of malignancy of the human prostate cancer.

Introduction

Progress in knowledge concerning the totality of human genes (genome) and production thereof in a given cell, tissue or organ (proteoma) has brought to light the extreme complexity of biological phenomena and regulation processes controlling them. At the same time, implementation of new biotechnological instruments which took place in the acquisition of molecular information forced the need for new instruments of analysis and management of experimental data for selection of significant and useful information in reaching understanding of a phenomena or process. Today numerous highly efficient methods are available which, while based on different principles, allow determination even simultaneously of the activity or presence of organic molecules in biological material. Therefore much information can be found by the researcher. In this scenario it therefore seems vitally important to determine which of these parameters and in which different combinations it is effectively informative and allows determining a biological phenomenon, and it appears ever more necessary to integrate together data coming from simultaneous and integrated measurement of an ever larger number of parameters. Recently the international scientific community reached consensus in estimating at several tens of thousands the genes present in the human genetic patrimony. It is imagined thus that, to represent and understand the overall picture of the functioning of the human machine in its entirety, just as understanding a particular pathological condition, it would be necessary to collect adequate information on the level of expression of all the genes (general profile of genic expression) which for various reasons condition the process to be studied since phenomena of extreme importance such as cellular differentiation and proliferation or neoplastic transformation can be considered ‘terminal products’ of a normal or pathological regulation of the total genic expression.

This systematic approach to the study of the genic expression can be conducted utilizing technologies which use microarray techniques. This can be considered the analytical phase of the study of complex biological phenomena. In this context, indeed, the conventional instruments of analysis appear largely insufficient to supply the enormous mass of information; hence the need for fully utilizing the potentialities offered by multidisciplinary integration and advanced biotechnologies for increasing to the utmost the sensitivity of the analysis and the acquisition capacity of the data. Maximum potential seems to be achievable today by the DNA array techniques on microchips. These methodologies, which require transversal competence in physics, chemistry, biochemistry, molecular biology and cellular and clinical medical biology offer extraordinary potentialities and diverse application possibilities but just because of their extreme sensitivity and strong dependence on the technology used they need to be validated in an unequivocal manner in the biological model studied. Implementation of these techniques thus passes through correct definition of the usual level of reliability, sensitivity and plasticity. This result can be achieved only by work integrating all the necessary competences which, often, come from disciplinary sectors not always accustomed to an intense exchange and a scientific dialectic.

It appears obvious that the research should express evaluations and reach conclusions that have a concrete effect. To this end it is necessary to carry out a careful critical analysis of the work done. Public opinion, investors and the researchers themselves seek to convert the potentialities into definite acquisition of reliable information such as to allow concrete quantifiable progress and produce tangible benefits. From the preliminary remarks it is evident that it is not a simple thing to adequately manage the great amount of information which the microarray techniques make available. On the one hand the role of bioinformatics appears crucial since it becomes indispensable both in the automation, acquisition and validation phase of the data and in the subsequent phase which passes from systematic analysis of the data to the final synthesis which originates the conclusions. On the other hand, experience, professionalism, creativity and specific qualification of the researcher continue to be always decisive for the good outcome of experimentation since in the ultimate analysis it is the professional researcher who draws the conclusions. Consequently, for the increase in complexity of the analysis there should be a correspondingly proportionate reduction in discretionary power and experimental error. In a subsequent phase the adequate synthesis of information, correct assessment of significant parameters and scientifically founded interpretative hypothesis will lead to pointing out of a minimal but statistically significant number of specific markers or molecular events necessary for characterizing a phenomenon, to thus give the starting signal to the applicative phase in which the possibility of determining a minimal package of molecules or the expression of a minimal number of genes becomes a concrete reality by means of the technique that proves most trustworthy and reproducible and most expedient from the economic point of view, and which, thanks to simple and expedient applicative protocols, can allow measurement of specific biological phenomena in a certain context. This information would be immediately applicable to molecular diagnostics, therapeutical monitoring and identification of new molecules having biological activity. To achieve this result, our work has allowed defining the minimal ‘genic expression patterns’ that identify, characterize and differentiate in an unequivocal manner the biological phenomenon of interest and also to evaluate whether integration of the genic expression data with those obtained by conventional methods might improve the predicative capability of the system. This is the purpose of the method presented here and showing how the qualifying element consists of the discovery of informative genes regardless of the study methodology used. This information can then be found either by applying known techniques like DNA microarray or Real-Time PCR or other methods being developed or that will become available in the future.

The method given here describes how informative data might be obtained by making use of the level of expression of a package of genes revealed by ourselves. In particular, this determination can be realized by use of the technique known as Real-Time PCR. For this purpose, the sequence of primers allowing said determination will be illustrated. But the intellectual property of any ensuing application of this method which, while based on even more efficient alternative methods, makes use of this knowledge for the purpose described above, is claimed here.

### DESCRIPTION

applications in the oncological field with particular reference to prostate cancer

1. General

Neoplastic conversion is a complex phenomenon which, although in some experimental models it has been shown how it might originate from a limited initial number of molecular events which carry out the role of promoters in its full-blown phase (and in clinical experience), it could be considered a pathology of the overall genic cell expression involving profound alterations of the metabolic network. In the majority of solid tumors this gives rise to a strong clonal heterogeneity and profound modifications of the structure of the chromosomes of the cells involved. In the preliminary remarks the need for having available adequate instruments for analysis of the molecular events in these complex systems was discussed. The prostate tumor (CaP) is a health problem of primary importance. Indeed, it is calculated that at the world level approximately 300,000 men develop prostate cancer each year, which places the impact of this neoplasia in fourth place among the most common in the world and in Western countries third place after lung cancer and colon-rectum cancer. This is an illness linked to age; with increase in life expectancy of the male population of the Western world this type of neoplasia is becoming a clinical and socioeconomic emergency of primary importance. Prospect studies foresee that it will become the first cause of death in the oncological field in adult males in coming years. Ever growing attention on the part of world scientific research and considerable biotechnological, human and economic resources are therefore set aside for this purpose to make this sector of research now one of the most competitive at the international level.

The CaP-applicable therapeutic possibilities have been rather limited heretofore; besides surgical operation and radiotherapy which are successful only in 40% of localized prostate tumors the antiandrogenic treatment still remains the only alternative therapy. Proposed and put into effect by Dr. Charles B. Huggins in the thirties (the discovery of CaP dependence on androgen hormones won the 1966 Nobel prize for Dr. Huggins), this therapy stimulated research on new molecules with antiandrogenic action and today it is applied in clinical practice with different strategies while taking advantage of a repertory of pharmacological nature which is after all rather modest. It should also be mentioned that antiandrogenic therapy allows at best only temporary CaP regression. Indeed, after a few years the tumor often starts to grow again because of development of the malignant cells whose growth is no longer inhibited by androgenic depletion (androgen-independent cells). For this clinical situation there is not available at present any remedy which might truly be called effective. This stage of the illness leads inexorably to progression of the neoplasia and relapse (‘hormonal escaping’), which usually appears with invasive character and development of bone metastasis fatal within 12 to 18 months.

Still today there is little knowledge about the biology of the prostate carcinoma and its molecular mechanisms leading to its onset and progression. None of the known oncogenics has yet been correlated unequivocally to development of this pathology. The only marker available for prostate cancer, prostate specific antigen (PSA) which is measured in the patient's plasma, has proven slightly reliable in precocious diagnosis since it does not discriminate with sufficient sensitivity between prostate hypertrophy (BPH) and CaP and in particular between forms of CaP with benign prognosis and forms with fatal prognosis (androgen-independent). In this framework the absolute necessity for having effective markers of the progression of the CaP is evident with particular reference to the development of refractoriness to anti-androgenic therapy. It is known that tumoral growth is a dynamic process whose progression is characterized in time by the relative number of both normal and tumoral cells subject to proliferation, death and quiescence. In particular, CaP is a heterogeneous illness whose polyclonality has already been shown. In addition, in cancerous tissue the transformed cells respond differently to hormonal environment and therapy, usually as a result of diffuse genetic alterations. This oncological model possesses characteristics of complexity such as to require the use of potent investigation techniques at the molecular level such as those discussed in the introductory remarks.

2. Experimental Models, Results Obtained and Method

The objective of validating analytical methods based on microarrays can be pursued simultaneously with in-depth analysis of the prostate physiopathology, a prerequisite for the study of the role of individual genes or groups of genes in the onset of androgen independence and neoplastic transformation.

The in vivo experimental model to which to make initial reference is the ventral prostate of a rat. This gland is subject to atrophy and involution following androgenic ablation by surgical castration. During involution of the prostate many psychopathological phenomena have been characterized among which variations of the proliferating capacity of the cells of the prostate epithelium, cellular atrophy, programmed cellular death (or apoptosis) and quiescence. Data obtained on some genes have shown their involvement on various grounds in these processes. Our studies, for example, have led to identification of a gene (clusterin, also known as SGP-2, TRPM-2, ApoJ, CLU and many other names and acrostics) whose expression increases enormously in the prostate of the rat subject to regression after collapse of the androgen levels following surgical or pharmacological castration (1-3). This gene, which is also found in man (4), is expressed in all the other organs or tissues and appears to be involved in numerous other physiopathologic processes suggesting that some degenerative processes leading to pathologies different by nature or localization can share some common molecular events. In particular its expression increases when the cells slacken their proliferation, suffer, die or become atrophic or quiescent (5-7).

Other genes are involved in these phenomena but for different reasons; some of them, like those controlling the metabolism of the aliphatic polyamine (ornithine decarboxylase, ODC; ornithine decarboxylase antizyme, OAZ; S-adenosyl-methionine decarboxylase, AdoMetDC; Spermidine/spermin N′-acetyltransferase, SSAT) are induced by the androgen hormones and their expression increases when the cells proliferate actively (2, 8, 9) or are converted into malignant cells. These genes, together with clusterin, are also involved in general phenomena like osmotic shock, stress, cellular differentiation and alteration of normal trophic relationships among the different types of cells in the tissue. Another class of genes involved are those which play a role in the cellular duplication process like histone H3. Genes like those belonging to the Growth arrest specific gene 1 (Gas1) class are induced in the cellular quiescence phase and show a proliferating block which can be accompanied by the state of distress of organs, tissues or cells. Genes regulating the glucidic metabolism among which glyceraldehyde 3-P dehydrogenase (GAPDH) are also involved. It is known that the ability to metabolize glucose under anaerobic conditions (anaerobic glycolysis) to produce lactic acid (lactic fermentation) is very important under conditions of tissue hypoxia (poor contribution of oxygen to the tissues), a condition which sets in usually in the initial development phases of a cancer before the phenomenon of production of new blood vessels (angiogenesis) allows irrigation of the tumoral mass.

For all these genes we have accumulated scientific evidence which shows their role not only as markers of phenomena but as causers of metabolic disturbances when their expression is altered or outside normal physiological control (10, 11). This information applies specifically to prostate cancer but, since the data obtained by this method describe phenomena of a more general character (cellular proliferation, cellular quiescence and proliferation arrest, cellular distress and apoptosis, cellular differentiation, glucidic metabolism, osmotic shock, response to stress, alteration of the normal trophic relationships between the different cellular types in the tissue et cetera), the information obtained by this method can also be applied in the characterization of all forms of neoplasia as well as tissue damage and repair, study of the response to treatment with drugs, and in the onset of resistance to pharmacological treatment (12). The data which we obtained also allow application of this method to renal (13, 14), cardiovascular (15) or neurodegenerative (6, 16) pathologies or the study of ageing (17, 18) or toxicity induced by heavy metals (19).

In the case of CaP, for the study of the stages of differentiation and transformation in the neoplastic sense, different in vitro experimental models are useable at present. Among these, primary cultures of normal, epithelial or connective pathological cells obtained from human or animal prostate and cellular lines of immortalized human or animal origin or with evident neoplastic characteristics which can be subjected to the action of hormones, trophic factors or drugs. The majority of the cellular lines used for the study of CaP mainly originate from the epithelium since it is generally here that this neoplasia develops. This biological material can be used to analyze the expression profile which characterizes the various stages of progression by applying the method described. The study can be performed either under conditions of basal growth or after administration of hormones, growth factors or medicines. The method can therefore be applied on cellular material obtained from patients. This allows studying the individual response of the patient to the different medicines to reach the choice of the most effective therapy in consideration of the fact that the CaP and all neoplasies in general are pathologies with strong individual connotation whose response to therapy is not always easy to predict.

This kind of approach arises again to describe and interpret the molecular stages leading to development of androgen-independent neoplasies under in vitro experimental conditions. Using these experimental models we obtained and confirmed much of the information discussed above (see the bibliography section on this point). The cell cultures, moreover, can be used as experimental test benches to verify the effect which manipulation of the genic expression of one or more genes of interest produces on the proliferative characteristics or the transformed phenotype. Simultaneous targeted manipulation of the genic expression of one or more genes can be obtained by transient or steady transfection using vectors of constitutive or inducible expression, mono- or polycistronic. This approach has already allowed us to produce useful data on the CaP (10, 11).

Going from the in vivo or in vitro experimental models discussed above in the study in surgical samples obtained from the operating room allowed us to verify both the utility of the information previously obtained and the plausibility of the formulated hypotheses by applying them to the clinical model. Using conventional experimental methods, we studied in tissue samples coming from human CaP a group of eight genes including:
A. Genes controlling the metabolism of the aliphatic polyamines
   1. Ornithine decarboxylase (ODC)
   2. Ornithine decarboxylase antizyme (OAZ)
   3. S-adenosyl-methionine decarboxylase (AdoMetDC)
   4. Spermidine/spermin N′-acetyltransferase (SSAT)
B. Marker genes for the proliferative for cellular state
   1. Histoneh3
   2. Growth-arrest specific gene 1 (Gas1)
C. Marker genes for androgen-dependence, cellular and apoptosis distress,
   1. Clusterin (SGP-2, ApoJ, TRPM-2, CLU)
D. Marker genes for glycolysis
   1. Glyceraldehyde 3-P dehydrogenase (GAPDH)

The group of genes was chosen on the basis of the information in our possession and for their involvement in proliferation, quiescence, neoplastic transformation, cellular differentiation, stress response, androgen-dependence and cellular distress phenomena. We were thus able to show that the level of expression of all these genes was modified in the malignant tissue in comparison with the corresponding healthy tissue obtained from the same patient, confirming that the neoplastic transformation process involves in general diffuse alterations of the genetic information that plausibly can be found in every form of cancer and in particular in CaP. Moreover, by standardizing and processing the data obtained by accurate measurement of their expression level by a statistical method which is an integral part of the method, it was possible to classify the degree of malignity of the CaP by using molecular criteria which proved to be more effective than conventional clinical and anatomopathological instruments (20). In particular, measurement of the expression level of these genes allowed discrimination between benign and neoplastic tissue (CaP analysis) and classification of cases of cancer as a function of the level of malignity (staging, characterization and typification of the CaP).

A follow-up lasting almost 5 years on patients included in the above study allowed us to correlate the expression level of the genes of our interest with the prognosis of the CaP. Despite the therapeutic operations of androgenic ablation and radical prostatectomy, more than 40% of the patients showed progression towards the aggressive form of the illness. Acquisition of the data, use of a statistical method which is an integral part of the method and combination of the molecular data obtained by ourselves with standard clinical data (degree and points according to Gleason, TNM stage, prostate volume, PSA value, age of patient, hereditary traits) according to different combinations led us to predict the prognosis of the patients with a precision not obtainable by conventional methods and correct classification of 100% of the patients with good prognosis and 90% of those with fatal prognosis with an overall average prediction of 95.7 of the patients studied. Subsequent studies have confirmed that expression of the clusterin is repressed prematurely in the transformed cells of the prostate while it is increased in the stroma surrounding the tumor (22). In addition, its expression increases when progress of the prostate cancer is inhibited by chemiopreventive agents (23). All this confirms the important role played by this gene in regulating proliferation of the prostate cells and constitutes scientific proof of the importance of determination of the level of expression of this gene, together with the others described above, for molecular characterization of the neoplastic transformation of the prostate cell and determination of the degree of malignancy and clinical prognosis.

3. Application Prospects

The data obtained from the above described study open new outlooks in the understanding of the behavior of CaP in early analysis, monitoring of the therapeutic response and clinical management, suggesting moreover possible new genetic targets for development of drugs or innovative therapeutic approaches. A first application of this method consists of determining the level of expression of a genes informative package made up of the 8 above-mentioned genes alone, in groups and in different associations. And all this regardless of the technique used. The data obtained thus are useful for choice and monitoring of the therapeutic approaches to be used and can be obtained from samples coming from the surgical room, from prostate needle biopsy or from biological material and fluid coming from prostate massage. The data obtained, alone, in groups or in different associations, integrated in different ways with the clinical information normally available in the department routine (degree and points according to Gleason, TNM stage, prostate volume, PSA value, age of patient and hereditary traits) allow early analysis, characterization and prediction of the malignity of the CaP after appropriate statistical analysis and processing of the data (CaP microarray) in accordance with a statistical method which is an integral part of the method. The data obtained thus are useful for choice and monitoring of the therapeutic approaches to be used and can be obtained from samples coming from the surgical room, from prostate needle biopsy or from biological material and fluid coming from prostate massage. In the future this method can be applied to haematic material also. Using micromanipulation techniques it is possible to take in a targeted manner samples consisting even of a few cells with characteristics clearly identified and homogeneous on the morphofunctional plane which can be subjected to molecular amplification techniques to obtain a quantity of material adequate for application of this method. Thanks to this method it is possible to face the difficulties deriving from the characteristics of heterogeneousness and polyclonality of the prostate tumor and increasing the sensitivity of the analysis. The method makes it possible to obtain the in vivo characterization (by prostate agobiopsia) of the neoplasia in the individual patient early to obtain a typification able to guide the therapeutic approach individually and which allows monitoring of the clinical case in real time.

Definition of the characteristic expression profiles (genic expression patterns) of the neoplastic transformation process in general and the CaP in particular, even for individuals, has also led to the discovery that the above mentioned genes which carry out an active roll in promoting and directing the tumoral progression are new genetic targets for new approaches and new applications of genic therapy.

### BIBLIOGRAPHY

1. Bettuzzi, S., Hiipakka, R. A., Gilna, P. and Liao, S. T. Identification of an androgen-repressed mRNA in rat ventral prostate as coding for sulphated glycoprotein 2 by cDNA cloning and sequence analysis, Biochem. J. 257; 293-296, 1989.
2. Bettuzzi, S., Zoli, M., Ferraguti, F., Ingletti, M. C., Agnati, L. F. and Corti, A. Regional and cellular distribution within the rat prostate of two mRNA species undergoing opposite regulation by androgens, J. Endocrinol. 132: 361-367, 1992.
3. Astancolle, S., Guidetti, G., Pinna, C., Corti, A., and Bettuzzi, S. Increased levels of clusterin (SGP-2) mRNA and protein accompany rat ventral prostate involution following finasteride treatment, J. Endocrinol. 167:197-204, 2000.
4. Purrello, M., Bettuzzi, S., Di Pietro, C., Mirabile, E., Di Blasi, M., Rimini, R., Grzeschik, K. H., Ingletti, C., Corti, A. and Sichel, G. The gene for SP-40,40, human homolog of rat sulfated glycoprotein 2, rat clusterin, and rat testosterone-repressed prostate message 2, maps to chromosome 8, Genomics. 10:151-156, 1991.
5.Bettuzzi, S., Troiano, L., Davalli, P., Tropea, F., Ingletti, M. C., Grassilli, E., Monti, D., Corti, A. and Franceschi, C. In vivo accumulation of sulfated glycoprotein 2 mRNA in rat thymocytes upon dexamethasone-induced celi death, Biochem. Biophys. Res. Commun. 175: 810-815, 1991.
6. Zoli, M., Ferraguti, F., Zini, I., Bettuzzi, S. and Agnati, L. F. Increases in sulphated glycoprotein-2 mRNA levels in the rat brain after transient forebrain ischemia or partial mesodiencephalic hemitransection, Brain Res. Mol. Brain Res. 18: 163-177, 1993.
7. Bettuzzi, S., Astancolle, S., Guidetti, G., Moretti, M., Tiozzo, R. and Corti, A. Clusterin (SGP-2) gene expression is cell cycle dependent in normal human dermal fibroblasts, FEBS Lett. 448; 297-300, 1999.
8. Grassilli, E., Bettuzzi, S., Monti, D., Ingletti, M. C., Franceschi, C. and Corti, A. Studies on the relationship between cell proliferation and cell death: opposite patterns of SGP-2 and ornithine decarboxylase mRNA accumulation in PHA-stimulated human lymphocytes, Biochem. Biophys. Res Commun. 180: 59-63, 1991.
9. Bettuzzi, S., Davalli, P., Astancolle, S., Pinna, C., Roncaglia, R., Boraldi, F., Tiozzo, R., Sharrard, M. and Corti, A. Coordinate changes of polyamine metabolism regulatory proteins during the cell cycle of normal human dermal fibroblasts, FEBS Lett. 446:18-22, 1999.
10. Scorcioni, F., Corti, A., Davalli, P., Astancolle, S. and Bettuzzi, S. Manipulation of the expression of regulatory genes of polyamine metabolism results in specific alterations of the cell-cycle progression, Biochem. J. 354:217-223, 2001.
11. Bettuzzi S., Scorcioni F., Astancolle S., Davalli P., Scaltriti M. and Corti A. Clusterin (SGP-2) transient overexpression decreases proliferation rate of SV40-immortalized human prostate epithelial cells by slowing down celi cycle progression, Oncogene, 21: 4328-4334, 2002.
12. Marverti, G., Bettuzzi, S., Astancolle, S., Pinna, C., Monti, M.G. and Moruzzi, M.S. Differential induction of spermidine/spermine N(1)-acetyltransferase activity in cisplatin-sensitive and resistant ovarian cancer cells in response to N(1),N(12)-bis(ethyl)spermine involves transcriptional and post-transcriptional regulation, Eur. J. Cancer. 37:281-289, 2001.
13. Bettuzzi, S., Mannelli, M., Strocchi, P., Davalli, P., Cevolani, D. and Corti A. Different localization of spermidine/spermine N1-acetyltransferase and ornithine decarboxylase transcripts in the rat kidney, FEBS Lett. 377:321-324,1995.
14. Bettuzzi, S., Strocchi, P., Davalli, P., Mannelli, M., Furci L. and Corti A. Androgen responsiveness and intrarenal localization of transcripts coding for the enzymes of polyamine metabolism in the mouse, Biochem. Celi Biol. 79: 133-140, 2001.
15. Pintus, G., Tadolini, B., Maioli, M., Posadino, A. M., Bennardini, F., Bettuzzi, S. and Ventura, C. Heparin inhibits phorbol ester-induced ornithine decarboxylase gene expression in endothelial cells, FEBS Lett. 423; 98-104,1998.
16. Zoli, M., Bettuzzi, S., Ferraguti, F., Ingletti, M. C., Zini, I., Fuxe, K., Agnati L. F. and Corti A. Regional increases in ornithine decarboxylase mRNA levels in the rat brain after partial mesodiencephalic hemitransection as revealed by in situ hybridization histochemistry, Neurochem. Int. 18: 347-352, 1991.
17. Bettuzzi, S., Strocchi, P., Mannelli, M., Astancolle, S., Davalli P. and Corti, A. Gene relaxation and aging: changes in the abundance of rat ventral prostate SGP-2 (clusterin) and ornithine decarboxylase mRNAs, FEBS Lett. 348; 255 - 258, 1994.
18. Mannelli, M., Quaglino, D., Bettuzzi, S., Strocchi, P., Davalli, P. and Corti, A. Increased levels of clusterin mRNA in the ventral prostate of the aging rat are associated to increases in cuboidal (atrophic) cell population and not to changes in apoptotic activity, Biochem. Celi Biol. 72; 515 - 521, 1994.
19. Davalli, P., Carpene, E., Serrazanetti, G. P., Bettuzzi, S., Viviani R. and Corti, A. Responses of poiyamine metabolism to metal treatment (Co, Cu, Zn, Cd) in the liver of the goldfish (Carassius auratus): distinct effects of season and temperature, Comp. Biochem. Physiol. 97C; 305-310, 1990.
20. Bettuzzi, S., Davalli, P., Astancolle, S. Carani, C., Madeo, B., Tampieri, A. and Corti, A. Tumor progression is accompanied by significant changes in the levels of expression of polyamine metabolism regulatory genes and clusterin (sulfated glycoprotein 2) in human prostate cancer specimens, Cancer Res. 60; 28-34, 2000.
21. S. Bettuzzi, M. Scaltriti, A. Caporali, M. Brausi, D. D'Arca, S. Astancolle, P.Davalli and A. Corti. Successful prediction of prostate cancer recurrence by gene profiling in combination with clinical data: a 5 years follow-up study, Cancer Research, 63, 3469-3472, 2003
22. M. Scaltriti, M. Brausi, A. Amorosi, G. Castagnetti, S. Astancolle, A. Corti, A. Caporali and S. Bettuzzi. Clusterin (SGP-2, ApoJ) expression is down-regulated in low and high grade human prostate cancer. Int. J. Cancer, 108, 23-30, 2004
23. A. Caporali, P. Davalli, S. Astancolle, D. D'Arca, M. Brausi, S. Bettuzzi and A.Corti. The chemopreventive action of catechins in the TRAMP mouse model of prostate carcinogenesis is accompanied by clusterin overexpression Carcinogenesis 2004, in press

1. A method for identification of neoplastic transformation with particular reference to prostate cancer by identification of a group of genes whose expression levels however determined even after integration with other data of clinical origin, proves informative for evaluation of the transformation of the tumoral transformation of prostate tissue, of its degree of malignancy and for prognosis of malignancy of human prostate cancer and characterized by understanding of the characteristic genie expression profile of genes belonging to classes A, B, C and D in which there are:
A. Genes controlling the metabolism of the aliphatic polyamines
1. Ornithine decarboxylase (ODC)
2. Ornithine decarboxylase antizyme (OAZ)
3. S-adenosyl-methionine decarboxylase (AdoMetDC)
4. Spermidine/spermin N′-acetyltransferase (SSAT) B. Marker genes for the cellular proliferative state
1. Histone H3
2. Growth-arrest specific gene 1 (Gas1)
C. Marker genes for androgen-dependence, cellular distress and apoptosis 24. Clusterin (SGP-2, ApoJ, TRPM-2, CLU)
D. Marker genes for glycolysis
1. Glyceraldehyde 3-P dehydrogenase (GAPDH) thanks to analysis made on biological samples independently of the determination methodology used.

2. The method in accordance with claim 1 consisting of obtaining the characteristic genie expression profile of the genes belonging to the above-mentioned classes A, B, C and D thanks to Real-Time PCR analysis making use of Primers for determination by Real-Time PCR of the above-mentioned informative genes in human tissues:

| | |
|---|---|
| Cas 1 | |
| DIR: | |
| 5′-CCC TGA CCC CCT ACC TGA-3′ | (SEQ ID NO: 1) |
| REV: | |
| 5′-CTT GGG CAT AGC CAG CAT GT-3′ | (SEQ ID NO: 2) |
| H3 | |
| DIR: | |
| 5′-CAG GAG GCT TGT GAG GCC TA-3′ | (SEQ ID NO: 3) |
| REV: | |
| 5′-AGC TGG ATG TCT TTG GGC AT-3′ | (SEQ ID NO: 4) |
| SSAT | |
| DIR: | |
| 5′-GGT TGC AGA AGT GCC GAA AG-3′ | (SEQ ID NO: 5) |
| REV: | |
| 5′-GTA ACT TGC CAA TCC ACG GG-3′ | (SEQ ID NO: 6) |
| Clusterin | |
| DIR: | |
| 5′-TGA TCC CAT CAC TGT GAC GG-3′ | (SEQ ID NO: 7) |
| REV: | |
| 5′-GCT TTT TGC GGT ATT CCT GC-3′ | (SEQ ID NO: 8) |
| ODC | |
| DIR: | |
| 5′-AGA CCT TCG TGC AGG CAA TC 3′ | (SEQ ID NO: 9) |
| REV: | |
| 5′-AGG AAA GCC ACC GCC AAT AT-3′ | (SEQ ID NO: 10) |
| AdoMet | |
| DIR: | |
| 5′-CAT CAC TCC AGA ACC AGA AT-3′ | (SEQ ID NO: 11) |
| REV: | |
| 5′-TAA CAA ACA AGG TGG TCA CA-3′ | (SEQ ID NO: 12) |
| OAZ | |
| DIR: | |
| 5′-CCT CCA CTG CTG TAG TAA CC-3′ | (SEQ ID NO: 13) |
| REV: | |
| 5′-GAA AGA TTG TGA TCC CTC TG-3′ | (SEQ ID NO: 14) |
| GAPDH | |
| DIR: | |
| 5′-AAC CTG CCA AAT ATG ATG AC-3′ | (SEQ ID NO: 15) |
| REV: | |
| 5′-TTG AAG TCA GAG GAG ACC AC-3′ | (SEQ ID NO: 16) |

3. The method in accordance with claim 1 consisting of the production of prognostic microchips based on DNA arrays consisting of the 8 above-mentioned genes alone, in groups and in differing associations.

4. The method in accordance with claim 1 consisting of the use of the data including the characteristic genic expression profile of the genes belonging to the classes A, B, C and D obtained by using Real-Time PCR analysis and the production of prognostic microchips based on DNA arrays consisting of the 8 genes either alone, in groups or in differing associations, integrated or not in different manners with the clinical information normally available in the department routine (degree and points according to Gleason, TNM stage, prostate volume, PSA value, age of patient, familiarity), to obtain the malignancy diagnosis, characterization of the prostate cancer (molecular typification) and prediction of malignancy (prognosis) of the CaP.

5. The method in accordance with claim 1 calling for manual or automatic data processing using standard statistical methods or an appropriate specific statistical analysis that is an integral part of the general method and allows correct interpretation of the data.

6. The method in accordance with claim 1 allowing applying the information obtained by the method described not only to prostate cancer; since the data obtained describing phenomena of a more general nature (cellular proliferation, cellular quiescence and proliferative arrest, cellular distress and apoptosis, cellular differentiation, glucidic metabolism, osmotic shock, stress response, alteration of the normal trophic relationships among the different cell types in the tissue, general metabolic responses and others), the information obtained by this method can also be applied in the characterization of all forms of neoplasia, tissue damage and repair, in the study of drug treatment response, in the onset of resistance to pharmacological treatment and in renal, cardiovascular and neurodegenerative pathologies, and in assessment of the state of ageing and toxicity induced by heavy metals.

7. The method in accordance with claim 1 calling for its application on any biological material and which can be used to analyze the expression profile of the genes described above to characterize the various neoplastic progression stages with the method being applicable on cellular material both in basal growth condition and after administration of hormones, growth factors and drugs with the method being applicable on cellular material obtained from patients for studying the individual response of said patient to the different drugs and reaching the choice of the most effective therapy, all in consideration of the fact that the CaP and all neoplasies in general are pathologies with strong individual connotation whose response to the therapy is not always easily predictable.

8. The method in accordance with claim 1 that can be used on samples coming from the surgery room, on prostate needle biopsies, on biological material and fluid coming from prostate massage and on haematic material for monitoring of the clinical case in real time.

9. The method in accordance with claim 1 applied to samples consisting even of a few cells with characteristics identified and homogeneous on the morphofunctional plane that can be subjected to molecular amplification techniques to obtain an adequate amount of material for studying the characteristics of heterogeneousness and polyclonality of the neoplasies with particular reference to the prostate tumor while increasing the sensitivity of the analysis.

10. The method in accordance with claim 1 leading to the identification of the genes belonging to classes A, B, C and D above-mentioned that perform an active role in promoting and addressing the tumoral progress as new molecular markers of the neoplastic progress and with particular reference to prostate cancer regardless of the method of study used.

11. The method in accordance with claim 1 leading to identification of the genes belonging to classes A, B, C and D above-mentioned that carry out an active role in promoting and addressing the tumoral progress as new genetic targets for new approaches and new applications of the gonic therapy of the neoplasies with particular reference to prostate cancer independently of the study method used.

12. The method in accordance with claim 1 comprising a statistical analysis that by converting the raw experimental data into standardized numbers makes allowance for individual and intra-experimental variations and assigns the level of significance in the prediction of phenomena of interest.
